(19) United States
(12) Plant Patent Application Publication
(10) Pub. No.: US 2003/0204885 P1
(43) Pub. Date: Oct. 30, 2003

(54) Strawberry plant named 'PLAROCIFRE'

(76) Inventor: Jose Miguel Arias Lopez, Tudela (ES)

Correspondence Address: CHRISTIE, PARKER & HALE, LLP, 350 WEST COLORADO BOULEVARD, SUITE 500, PASADENA, CA, 91105, US

(21) Appl. No.: 10/128,893

(22) Filed: Apr. 24, 2002

(51) Int. Cl. A01H 5/00

(52) U.S. Cl. PLT/208

(57) ABSTRACT

A new strawberry plant variety characterized by medium plant vigor and density and very early as well as abundant production of red colored, conical shaped and firm, large-size fruit.

### BACKGROUND OF THE INVENTION

The new variety of strawberry was created in a breeding program by crossing two parents; in particular, by crossing as seed parent a variety designated 9261 and as pollen parent a variety designated 86-032, both undistributed seedlings. Both parental varieties are proprietary and have not been commercialized.

The seedling of the new variety was grown and asexually propagated by runners in Soria, Spain, 3 W., 41 N., 3000 feet elevation. Clones of the new variety were further asexually propagated and extensively tested. This propagation and testing has demonstrated that the combination of traits disclosed herein which characterize the new variety are fixed and retained true to type through successive generations of asexual reproduction.

### SUMMARY OF THE INVENTION

The present invention relates to a new and distinct strawberry variety. The varietal denomination of the new variety is ‘PLAROCIFRE.’ Among the characteristics which distinguish the new variety from other varieties are a combination of traits which include medium plant vigor and density and very early and abundant production of red colored, conical-shaped, firm and large fruit.

### COMPARISON TO CLOSEST VARIETY

The new variety closest to the variety ‘CAMAROSA’ (U.S. Plant Pat. No. PP8,708), but is distinguished therefrom by the following characteristics possessed by ‘PLAROCIFRE’ which are different than, or not possessed by, ‘CAMAROSA.’

1. ‘CAMAROSA’ exhibits more plant vigor and more plant density than ‘PLAROCIFRE.’

2. Length/width ratio of the terminal leaflet in ‘CAMAROSA’ is as long as broad, whereas in ‘PLAROCIFRE’ it is longer than broad.

3. Shape of base of the terminal leaflet in ‘CAMAROSA’ is obtuse, whereas in ‘PLAROCIFRE’ it is acute.

4. ‘CAMAROSA’ exhibits more large fruits than ‘PLAROCIFRE.’

5. ‘CAMAROSA’ has fruit with almost cylindrical shape, whereas fruit of ‘PLAROCIFRE’ is conical.

6. ‘CAMAROSA’ fruit is a dark red color (near 47B to 47A), whereas in ‘PLAROCIFRE’, fruit color is red (near 43A to 43B).

7. ‘CAMAROSA’ fruits have a dark red flesh color (near 42A), whereas in ‘PLAROCIFRE’ fruits have light red flesh color (near 42C to 40C).

8. Insertion of calyx in the fruits of ‘CAMAROSA’ is level, whereas in ‘PLAROCIFRE’ it is set above the fruit.

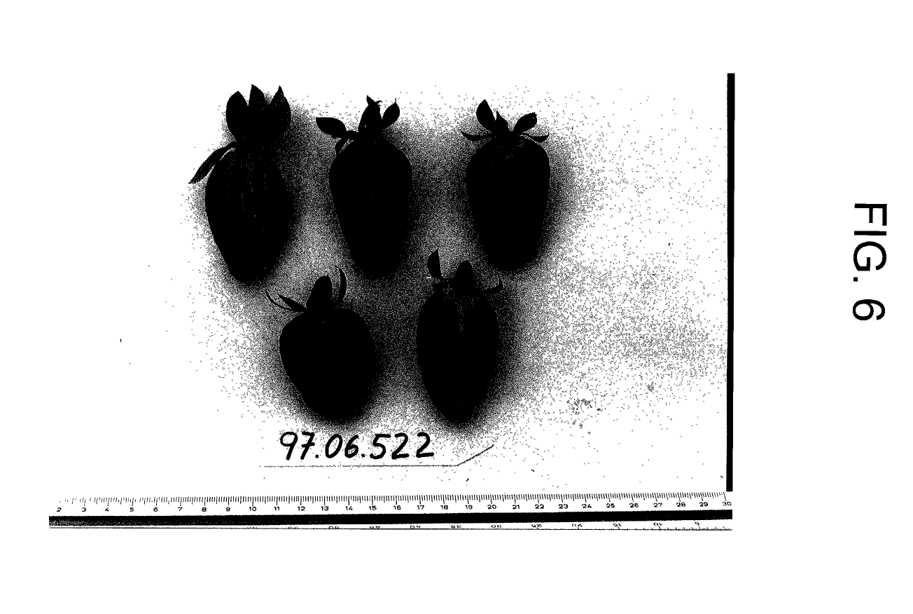

The differences in the fruits of ‘CAMAROSA’ and the new variety are shown in FIGS. 6 and 7, and FIGS. 8 and 9. These differences are maintained during the harvest season.

9. Time of flowering in ‘PLAROCIFRE’ is earlier than in ‘CAMAROSA.’

10. Time of ripening in ‘PLAROCIFRE’ is earlier than in ‘CAMAROSA.’

### BRIEF DESCRIPTION OF ILLUSTRATIONS

The accompany photographs show typical specimens of the new variety (designated 97.06.522) in the illustrations, including fruit, foliage and flower, in color as nearly true as it is reasonably possible to make in color illustrations of this character.

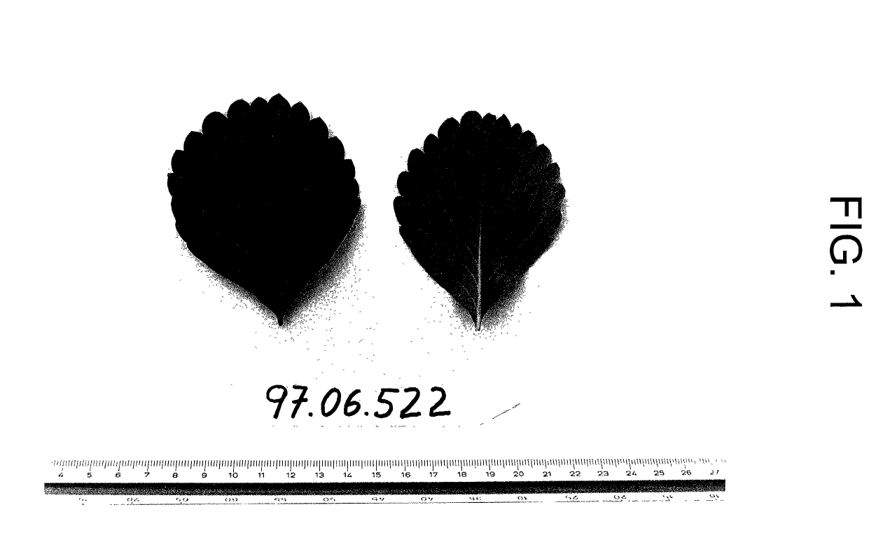

FIG. 1 shows the upper side and underside of a typical foliage of the new variety (designated 97.06.522), with an acute base shape and in which the length/width ratio is longer than broad.

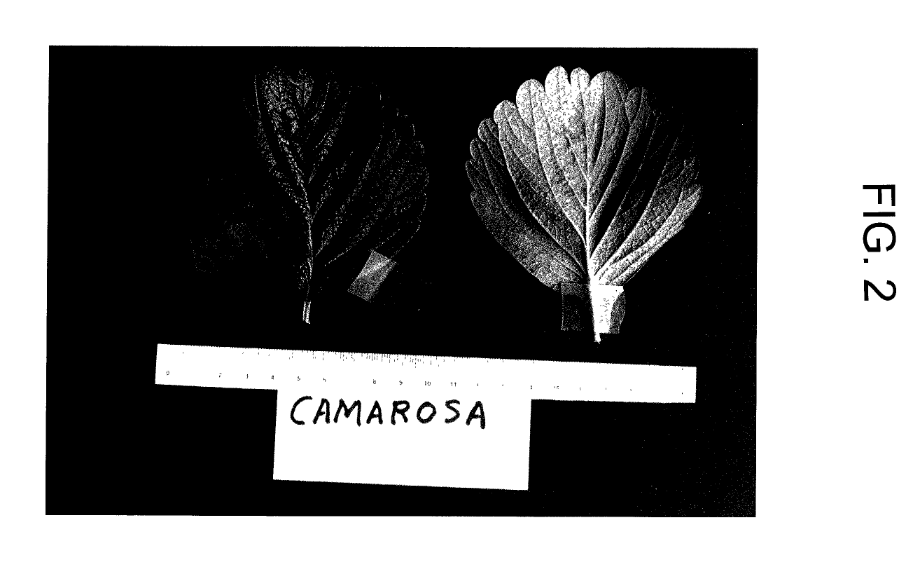

FIG. 2 shows the upper side and underside of a typical terminal leaflet of ‘CAMAROSA’ with an obtuse base shape and in which the length/width ratio is as long as broad.

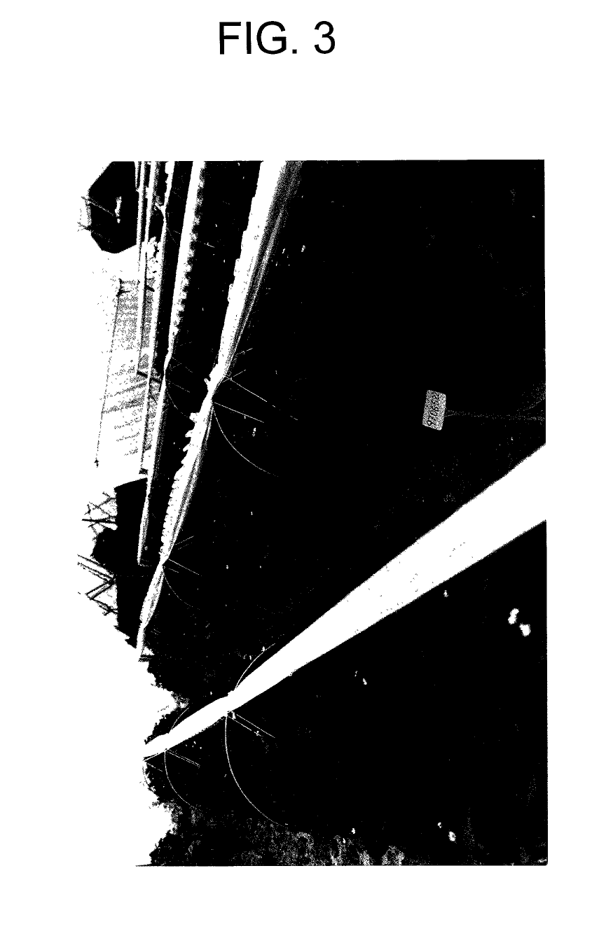

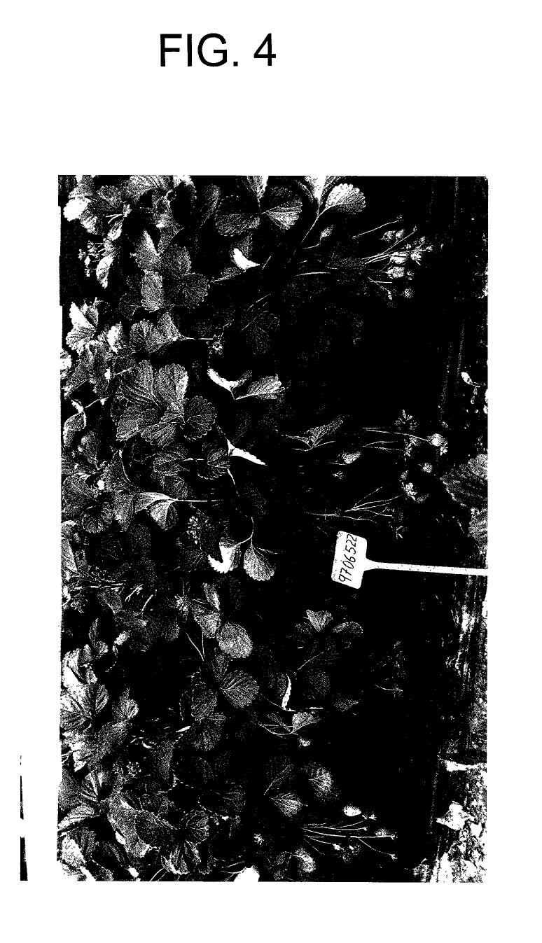

FIGS. 3 and 4 show the fruit against a background of the upper side of the foliage of the new variety (designated 97.06.522).

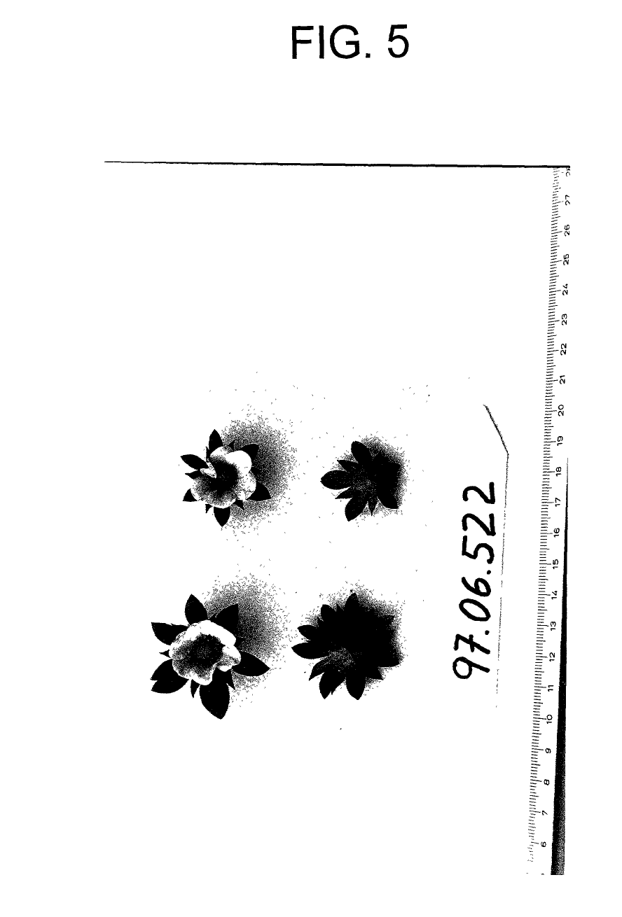

FIG. 5 shows the flower and reproductive organs of the new variety (designated 97.06.522).

FIG. 6 shows typical fruits of the new variety (designated 97.06.522) in cross section illustrating the typical flesh and flesh coloration, conspicuous core and core cavity and conical shape and the insertion of calyx in the fruits is set above fruit.

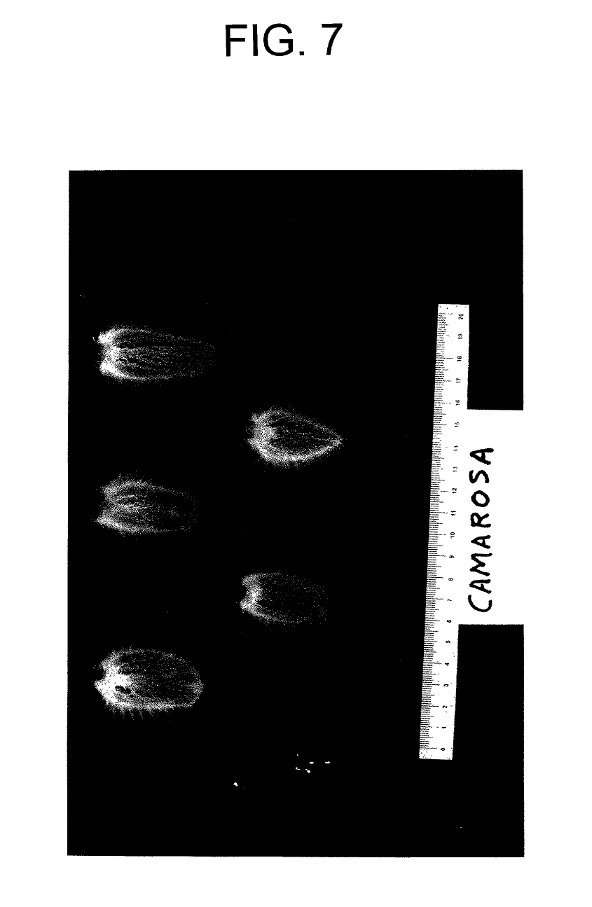

FIG. 7 shows typical fruits of the ‘CAMAROSA’ in cross section illustrating the typical flesh and flesh coloration, conspicuous core and core cavity and almost cylindrical shape and the insertion of calyx in the fruits is level.

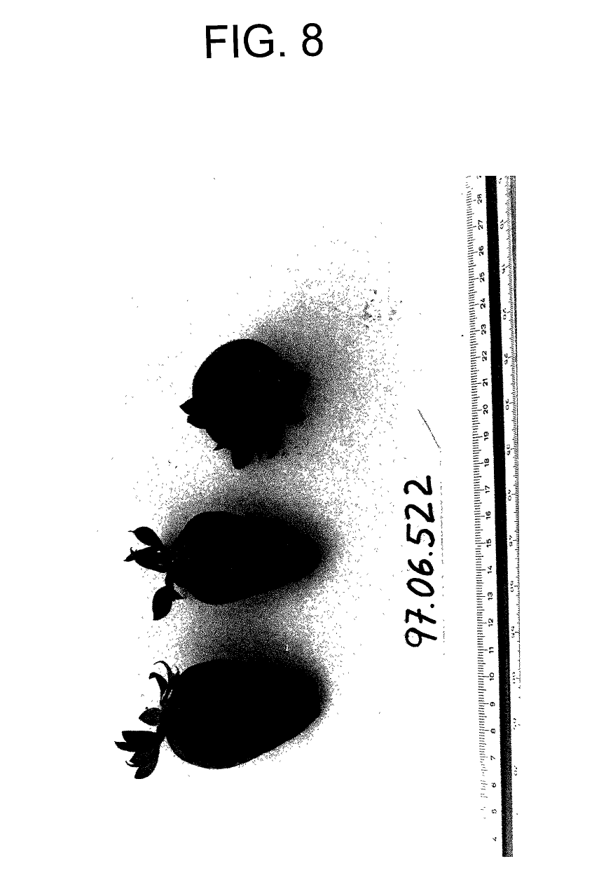

FIG. 8 shows whole and sliced, detached fruit of the new variety (designated 97.06.522) with the typical red color.

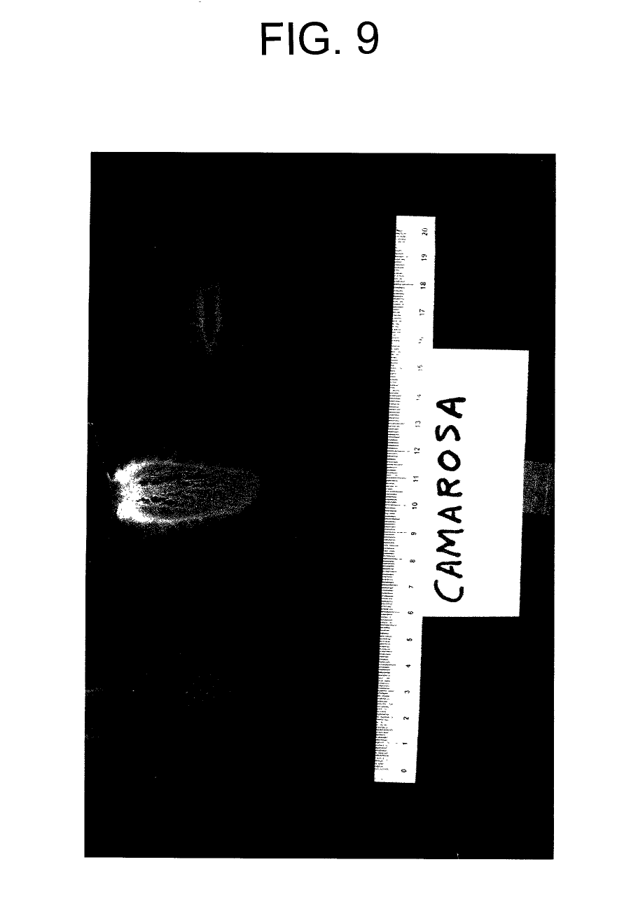

FIG. 9 shows whole and sliced, detached fruit of the ‘CAMAROSA’ with the typical red dark color.

### DESCRIPTION OF THE NEW VARIETY

The following detailed description of the new variety is based upon observations taken of plants and fruits grown “underglass,” i.e. undertunnel, in the farm of La Mogalla in Cartaya (Huelva), Spain, 7° W., 37° N., 45 feet elevation.

The following description is in accordance with UPOV terminology and the color terminology herein is in accordance with The Royal Horticultural Society Colour Chart (R.H.S.C.C.). The color descriptions and other phenotypical descriptions may deviate from the stated values and descriptions depending upon variation environmental, seasonal, climatic, and cultural conditions.

### PROPAGATION

The new variety is principally propagated by way of runners. Although propagation by runners is presently preferred, other know methods of propagating strawberry plants may be used. Strawberries root well after transplanting.

The term “blistering” used herein refers to the texture or rugosity or surface ondulation inherent to leaves and is generally a constant characteristic.

### GENERAL

‘PLAROCIFRE’ is a short day variety that needs an induction to flowering by chilling, such as occurs at a high elevation nursery (fresh plant) or with cold storage (referred to as a frigo). Usually a short time is sufficient. ‘PLAROCIFRE’ is self-fertile. It produces a large quantity of pollen throughout the seasons and pollination is generally good as there are very few malformed fruit.

Production: Plants described are from high elevation nursery.

Trials pursued in Cartaya (Huelva), Spain.

Date of planting: 23rd Oct. 2000.

Number of repetitions: 2

Plants per repetition: 150

Comparison with ‘CAMAROSA’: The new variety is compared with ‘CAMAROSA’ in FIG. 1 with 2 and FIG. 6 with 7 and FIG. 8 with 9.

Accumulated production of 1^{st} quality fruit (g/plant)

| Variety | 23-Fb | 28-Mr | 24-Ap | 15-My |
|---|---|---|---|---|
| ‘CARTUNO’ | 2 | 135 | 528 | 719 |
| ‘CAMAROSA’ | 8 | 181 | 624 | 861 |
| ‘TUDNEW’ | 26 | 198 | 544 | 676 |
| ‘PLAROCIFRE’ | 30 | 225 | 503 | 706 |

| | 1^{st} + 2^{nd} | | Weight |
|---|---|---|---|
| Variety | Quality Fruit | Total | (g/fruit) |
| ‘CARTUNO’ | 719 + 111 | 830 | 22-21 |
| ‘CAMAROSA’ | 861 + 157 | 1018 | 24-23 |
| ‘TUDNEW’ | 676 + 86 | 762 | 24-23 |
| ‘PLAROCIFRE’ | 706 + 79 | 785 | 24-23 |

Production total, to 15 May,
of First Quality Fruit(1^{st} quality)
and Second Quality Fruit (2^{nd} quality)
in g/plant

| | | | TOTAL | |
|---|---|---|---|---|
| | 1^{st} | 2^{nd} | (1^{st} quality + | % 2^{nd} |
| Variety | quality | quality | 2^{nd} quality) | quality |
| CARTUNO | 719 | 111 | 830 | 13 |
| CAMAROSA | 861 | 157 | 1018 | 15 |
| TUDNEW | 676 | 86 | 762 | 11 |
| PLACARTFRE | 766 | 88 | 854 | 10 |

$\% {2}^{nd} quality = \frac{{2}^{nd} quality}{TOTAL} \times 100$

Weight (g/Fruit) at two dates: 28 March and 15 May

| Weight (g/fruit) | 28 March | 15 May |
|---|---|---|
| ‘CARTUNO’ | 22 | 21 |
| ‘CAMAROSA’ | 24 | 23 |
| ‘TUDNEW’ | 24 | 23 |
| ‘PLAROCIFRE’ | 24 | 23 |

WEIGHT is shown as the average weight per fruit in First Quality Fruits.

FRUIT ANALYSIS

| | ‘CAMA- | ‘PLAROCIFRE’ | | |
|---|---|---|---|---|
| | ROSA’ | (97.06.522) | ‘CARTUNO’ | ‘TUDNEW’ |
| Firmness | 1.20 | 1.20 | .70 | .90 |
| (KG) | | | | |
| Humidity | 92.80 | 89.90 | 93.30 | 88.00 |
| & Volatile | | | | |
| Matter | | | | |
| (%) | | | | |
| Dry | 7.20 | 10.10 | 6.70 | 12.00 |
| Matter | | | | |
| (%) | | | | |
| PH (to | 3.50 | 3.70 | 3.90 | 3.60 |
| 20°) | | | | |
| Acidity as | .80 | .80 | .51 | .90 |
| Anhydride | | | | |
| Citric (%) | | | | |
| Soluble | 9.50 | 10.40 | 10.90 | 8.60 |
| Solids | | | | |
| (°Brix) | | | | |
| Maturity | 11.90 | 12.70 | 21.40 | 9.60 |
| Index | | | | |
| Ascorbic | 500 | 620 | 560 | 820 |
| Acid | | | | |
| Content | | | | |
| (ppm) | | | | |
| Dominant | 495 | 490 | 495 | 495 |
| Tonality | | | | |
| (nm) | | | | |
| Lumi- | | | | |
| nosity: | | | | |
| Trans- | 26.50 | 24.80 | 30.70 | 16.70 |
| mitance to | | | | |
| 460 nm | | | | |

The following definitions apply:

Firmness: It is the fruit's resistance to penetration measured in Kilograms (Kg). The measure given has been obtained by the penetrometer ROZE Mod. Arbelette, with a 50 mm² section head.

Dry Matter: It is the weight of the residual left from the trituration of the fruit after the drying process at a temperature of 103° C.+2° C. until reaching constant weight.

$$( \% ) Dry Matter = \frac{Weight Dry Matter}{Weight Fresh Matter} \times 100$$

Humidity & Volatile

Matter: Represents the content in volatile matters and water of the fruits.

(%) Humidity & Volatile Matter=100−% Dry Matter

Maturity Index: Relation between Soluble solids and Acidity as Anhydride Citric.

$$Maturity Index = \frac{Soluble solids}{Acidity as Anhydride Citric}$$

The following additional information is provided to further describe the new variety:

### VARIETY: ‘PLAROCIFRE’ Breeder Ref. 97.06.522

Classification: Fragaria, L.

### PLANT

Habit: Flat globose

Density: Medium

Vigor: Medium

Height: About 17 cm

Width: About 24 cm

### LEAF

Color: Upper side near 138A to 139A; underside near 139D to 141D

Length: About 10 cm

Width: About 11 cm

Cross section: Slightly concave

Leaf surface undulation or

Blistering: Weak

Number of leaflets: Three (3) only

Leaf stem characteristics Length about 10 cm

Color: Near 138D

Position of hairs: Upwards

Terminal leaflet

Length/width ratio.—Longer than broad.

Length.—About 7 cm.

Width.—About 6 cm.

Shape of base.—Acute.

Shape of teeth.—Obtuse.

Petiole Length about 10 cm

Position of hairs.—Upwards.

Stipule: Anthocyanin coloration — weak

Stolons Medium, about 7

Anthocyanin coloration.—Medium, near 142D.

Thickness.—Medium, about 3 mm.

Pubescence.—Medium.

### INFLORESCENCE

Position relative to foliage: Level with

Flower

Size.—Medium.

Size of calyx relative to corolla.—Larger.

Size of inner calyx relative to outer.—Larger.

Spacing of petals.—Overlapping.

Flower characteristics

Primary flowers.—Diameter about 3.5 cm.

Secondary flowers.—Diameter about 3 cm.

Number of petals.—Normally about 6 — no significant fragrance.

Time from bloom to mature fruit (in Huelva, Spain).—About 35 to 40 days.

Petal.—Length/Width ration: As long as broad Attitude semi-erect.

### FRUITING TRUSS Attitude semi-erect

### FRUIT

Ration of length/maximum width: Longer than broad

Color: Near 43A to 43B

Peduncle length of inflorescence stem: Primary fruit about 9 cm to 11 cm, secondary fruit about 6 cm to 7 cm, color near 138D

Primary fruit

Length.—About 6 cm.

Width.—About 4.5 cm.

Secondary fruit

Length.—About 4.5 cm.

Width.—About 3 cm.

Size.—Large.

Predominant shape.—Conical.

Difference in shapes between primary and secondary fruits: Slight

Bank without achenes: Narrow

Unevenness of surface: Weak

Color: Near 43A to 43B

Evenness of color: Even

Glossiness: Strong

Insertion of achenes: Level with surface

Insertion of calyx: Set above fruit

Pose of the calyx segments: Reflexed

Size of calyx in relation to fruit diameter: Larger

Adherence of calyx: Medium

Firmness: Very firm

Color of flesh: Medium red, near 42C to 40C; lightening toward center

Evenness of color of flesh: Slightly uneven

Sweetness: Medium

Acidity: Medium

Time of flowering: Early

Time of ripening: Early

Type of bearing: Not remonstrant

Chilling: Weak

Planting date: Oct. 23, 2000

10% flowering: Dec. 12, 2000

First mature fruits: Jan. 23, 2001

Maturity (15-20 gms/plant): Feb. 10, 2001

Time of flowering date: Date of planting: Oct. 23, 2000 in the farm of La Mogalla, in Cartaya (Huelva), Spain, about 7° W, 37° N, 45 feet elevation. Ten (10%) percent flowering occurs about Dec. 12, 2000 with first mature fruit about Jan. 23, 2001 and maturity (15-20 g/plant) about Feb. 10, 2001.

Time of flowers (50% of plants at first flower): about Dec. 22, 2000.

Storage Qualities: ‘PLAROCIFRE’ fruit maintain their quality characteristics when keeping them in a frigo chamber at temperatures of about 2° C. during 48 hours. The fruit's color remains substantially the same.

Time of ripening: After planting as aforesaid, plants are grown in raised beds under tunnel (small tunnel with small holes in plastic walls). Water and fertilizer were applied through drip irrigation. Time of ripening (50% of plants with ripe fruit) is about January 29^{th}. First mature fruit is about January 23^{rd} and maturity (15-20 gms/plant) is about February 10^{th}.

General: The growing period in Huelva, Spain, where the observations were made is between about December 10^{th} and May 15^{th} of each year, with a maximum production at about mid-April. ‘PLAROCIFRE’ is a short variety that benefits from induction to flowering by chilling, usually a few hours sufficient, preferably at temperatures of 7° C. or less. Normally, the minimum number of hours is accumulated in the field during several days.

Disease resistance: No particular sensitivity to any disease or parasite has been observed for ‘PLAROCIFRE’.

I claim:

1. A new and distinct strawberry plant of the variety substantially as shown and described.
